(19) United States
(12) Patent Application Publication
(10) Pub. No.: US 2023/0386642 A1
(43) Pub. Date: Nov. 30, 2023

(54) COMPUTER-BASED WELL BEING ASSESSMENT AND MITIGATION

(71) Applicant: Microsoft Technology Licensing, LLC, Redmond, WA (US)

(72) Inventor: Mastafa Hamza FOUFA, Nimes (FR)

(73) Assignee: Microsoft Technology Licensing, LLC, Redmond, WA (US)

(21) Appl. No.: 17/664,974

(22) Filed: May 25, 2022

(51) Int. Cl. G16H 20/70; G16H 10/20; G16H 40/67; G16H 50/20; G06V 40/16; G10L 25/63

(52) U.S. Cl. CPC G16H 20/70; G16H 10/20; G16H 40/67; G16H 50/20; G06V 40/174; G10L 25/63

(57) ABSTRACT

Examples are disclosed that relate to performing computer-based well being assessments and proactively performing computer-based mitigation operations to improve a user's well being. In one example, a computing system comprises a network communication subsystem, an attribution machine, a well being assessment machine, and a mitigation machine. The network communication subsystem is configured to communicate with a plurality of user computers. The attribution machine is configured to attribute, to a user account, computing information that the network communication subsystem receives from a user computer. The well being assessment machine is configured to progressively update a well being score over time for the user account based at least on the computing information. The mitigation machine is configured to perform a mitigation operation associated with the user account based at least on an above threshold rate of decrease of the well being score.

### BACKGROUND

Due to advances in computer technology, human interactions that once existed only in-person now can be conducted virtually using computers. For example, work, meetings, educational classes, conversations, and chats all may be conducted virtually. However, in some cases, the flexibility provided by the ability to interact virtually has produced less opportunity for people to have regular in-person interactions. The resulting situation causes some people to feel isolated and disconnected from other people, which affects those people's well being.

### SUMMARY

Examples are disclosed that relate to performing computer-based well being assessments and proactively performing computer-based mitigation operations to improve a user's well being. In one example, a computing system comprises a network communication subsystem, an attribution machine, a well being assessment machine, and a mitigation machine. The network communication subsystem is configured to communicate with a plurality of user computers. The attribution machine is configured to attribute, to a user account, computing information that the network communication subsystem receives from a user computer. The well being assessment machine is configured to progressively update a well being score over time for the user account based at least on the computing information. The mitigation machine is configured to perform a mitigation operation associated with the user account based at least on an above threshold rate of decrease of the well being score.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

FIG. 1 shows an example scenario in which a user is interacting with a computer, and the computer generates computing information based at least on such interactions to assess the user's well being.

FIG. 2 shows an example computing system that is configured to assess a user's well being and selectively perform mitigation operations to improve the user's well being.

FIG. 3 shows an example user computer configured to generate user-specific computing information to assess a user's well being.

FIG. 4 shows a plurality of example scenarios in which users having different well being scores are handled differently based at least on different rates of change of the well being scores.

FIG. 5 shows an example social network graph of a user including users that have positive interaction qualities and users that have negative interaction qualities.

FIGS. 6-8 show example notifications that may be generated as part of mitigation operations performed to improve a user's well being.

FIGS. 9-10 show an example computer-implemented method for assessing a user's well being.

FIG. 11 shows an example computing system.

### DETAILED DESCRIPTION

The present description is directed to a well being assessment approach in which quantitative computer-based well being assessments are performed and computer-based mitigation operations are performed based at least on such well being assessments in order to proactively improve a user's well being. In particular, one or more well being assessment artificial intelligence (AI) machines are configured to perform computer-based well being assessments.

The well being assessment AI machine(s) are configured to receive computing information based at least on user interactions with various computer application programs and output a well being score for a user based at least on the computing information. Furthermore, the well being assessment AI machine(s) are configured to progressively update a user's well being score over time to detect changes in the user's well being.

The well being assessment AI machine(s) are configured to perform one or more mitigation operations based at least on an above threshold rate of decrease in the user's well being score. The mitigation operation(s) are performed in computer application programs with which the user interacts. For example, the mitigation operation(s) may include actionable steps that prompt a user to connect with other people or prompt other people to reach out and connect with the user in order to improve the user's well being.

By using the rate of decrease of the well being score as the trigger for performing the mitigation operations, a distinct change in the user's behavior can be accurately identified before intervening and providing mitigation to improve the user's well being. Such an approach may be more accurate than acting on general or snapshot assessments of a user's well being that do not track how a user's well being is changing over time. As used herein, “rate” may refer to an instantaneous rate and/or an average rate over a certain period of time.

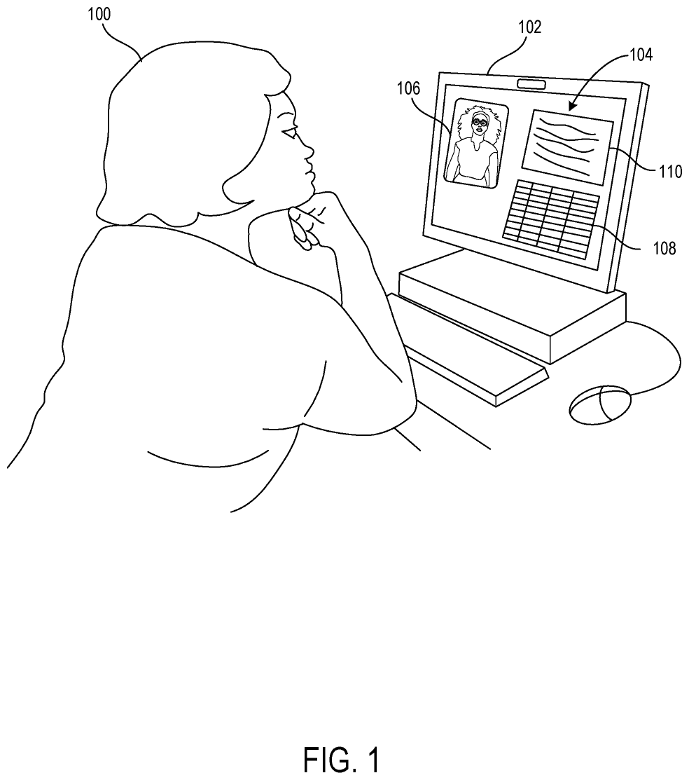

FIG. 1 shows an example scenario where a user 100 is interacting with a plurality of different computer application programs executed by a user computer 102. The plurality of computer application programs includes personal communication application programs and productivity application programs. For example, the user 100 is having a conversation with a second user via a personal communication application program in the form of a video conferencing application program 106. At the same time, both of the users are collaborating on a shared spreadsheet generated by a spreadsheet application program 108, which is an example of a productivity application program. Additionally, as another example of a productivity application program, an email application program 110 is executing on the user computer 102, so that the user 100 can monitor incoming email messages.

The user computer 102 is configured to track each of these user-specific interactions by collecting, in strict accordance with user-authorized privacy settings, computing information that is specific to the user for purposes of assessing the user's well being. The computing information may include a range of different types of information, which may be anonymized or pseudo-anonymized in accordance with user-authorized privacy settings. Such information may include raw data, parameters derived from the raw data, and/or user-state metrics that are derived from the parameters/raw data.

Whenever user information is collected for any purpose, the user information is collected with the utmost respect for user privacy (e.g., user information is only collected after the user owning the information provides affirmative consent). Whenever information is stored, accessed, and/or processed, the information is handled in accordance with privacy and/or security standards to which the user has opted in. Prior to user information being collected, users may designate how the information is to be used and/or stored, and user information may only be used for the specific, objective-driven purposes for which the user has opted in. Users may opt-in and/or opt-out of information collection at any time. After information has been collected, users may issue a command to delete the information, and/or restrict access to the information. All potentially sensitive information optionally may be encrypted and/or, when feasible anonymized or pseudo-anonymized, to further protect user privacy. Users may optionally designate portions of data, metadata, or statistics/results of processing data for release to specific, user-selected other parties, e.g., for further processing. Information that is private and/or confidential may be kept completely private, e.g., only decrypted temporarily for processing, or only decrypted for processing on a user device and otherwise stored in encrypted form. Users may hold and control encryption keys for the encrypted information. Alternately or additionally, users may designate a trusted third party to hold and control encryption keys for the encrypted information, e.g., so as to provide access to the information to the user according to a suitable authentication protocol.

In the example illustrated in FIG. 1, computing information collected for the user may include comments posted in the spreadsheet generated by the spreadsheet application program 108, email messages sent by the user 100 via the email application program 110, as well as content of utterances spoken by the user, voice patterns, and facial expression patterns collected via the video conferencing application program 106, as nonlimiting examples.

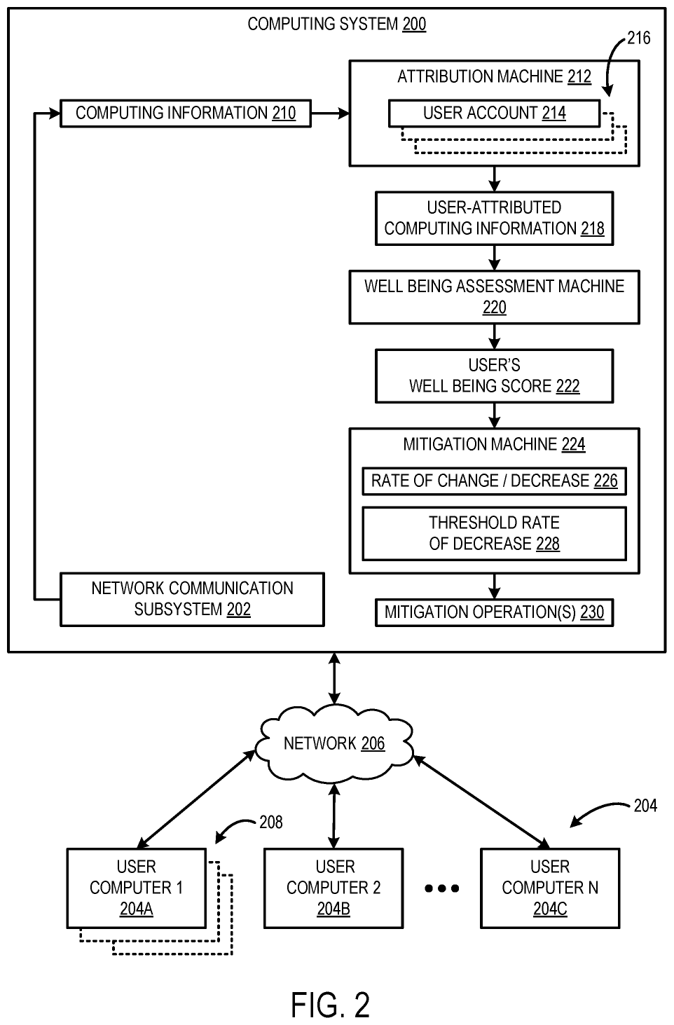

Such tracking can be performed through the application programs themselves, an operating system, and/or other activity tracking services of the user computer 102. The user computer 102 is configured to send the computing information to a computing system 200 (shown in FIG. 2) via a computer network. The computing system 200 is configured to perform quantitative well being assessments for a plurality of different users based at least on computing information received from a plurality of different user computers associated with the plurality of different users. The computing system 200 is further configured to selectively perform mitigation operations based at least on such well being assessments in order to proactively improve the well being of the plurality of different users as will be discussed in further detail below.

The user computer 102 is provided as a non-limiting example of a computer that is associated with a user, e.g., via a user account, and is configured to facilitate computer-based assessment of the user's well being. The concepts discussed herein are broadly applicable to any suitable type of user computer or computing system including a laptop computing device, a mobile computing device (e.g., smartphone), a wearable computing device, a mixed/augmented/virtual reality computing device, or another type of user computer.

FIG. 2 shows the computing system 200 that is configured to assess user well being and selectively perform mitigation operations to improve the user well being. The computing system 200 includes a network communication subsystem 202 configured to communicate with a plurality of user computers 204 (e.g., first user computer 204A, second user computer 204B, Nth user computer 204C) via a computer network 206. The different user computers 204 are associated with different users, e.g., via a user account. In one example, the first user computer 204A may correspond to the user computer 102 associated with the first user 100 shown in FIG. 1. The second user computer 204B may be associated with a second user, and the Nth user computer 204C may be associated with a third user. The computing system 200 may be configured to communicate with any suitable number of different user computers (e.g., 1-N) via the network communication subsystem 202.

In some examples, a plurality of user computers 208 may be associated with the same user. For example, a work desktop computer, a home desktop computer, a laptop computer, a tablet, a smartphone, a smart watch, and a game console may be associated with the same user. User-specific interactions may be tracked, and user-specific computing information may be collected across any or all user computers 208 associated with a particular user. Any suitable number of user computers may be associated with a particular user.

The computing system 200 is configured to receive, via the network communication subsystem 202, computing information 210 from the plurality of user computers 204. In some examples, the computing system 200 additionally or alternatively may receive the computing information 210 from other types of computers. In one example, the computing information 210 may be received from a cloud service computer that collects/tracks user-specific computing information based at least on user interactions with application programs executing on a user computer.

The computing system 200 includes an attribution machine 212 configured to attribute the computing information 210 that the network communication subsystem receives from a user computer of the plurality of user computers 204 to a user account 214 of a plurality of user accounts 216 associated with different users. In other words, the attribution machine 212 is configured to determine that computing information 210 is specific to a particular user and attribute the computing information to the user account 214 associated with the user. The attribution machine 212 allows the computing system 200 to process computing information for the plurality of different user accounts 216 as the computing information is received from the plurality of different user computers 204.

The attribution machine 212 may be configured to attribute the computing information 210 to a user account of the plurality of user accounts 216 in any suitable manner. In some examples, a user will be required to login using verifiable credentials (e.g., username and password), thus facilitating attribution. In one example, the attribution machine 212 may be configured to associate a computer IDs (e.g., IP addresses) of user computer that sent the computing information with a user account and attribute the computing information with the user account based at least on the association. In another example, the computing information attribution machine 212 may be configured to interpret metadata associated with the computing information with a user account and attribute the computing information with the user account based at least on the metadata. In some examples, attributing computing information with a user account may include storing the computing information as an entry in a database or another data storage mechanism, such that the computing information may be available and accessible for use in processing operations related to assessing a user's well being.

The computing information 210 may take any suitable form. In some examples, the computing information 210 may include raw data generated by application programs executing on a user computer. In some examples, the computing information 210 may include parameters or metrics that are generated by a user computer. The parameters or metrics may be derived from or distilled down from the raw data. Such parameters or metrics may provide indications of higher-level aspects of a user's state of well being and/or changes in a user's behavior.

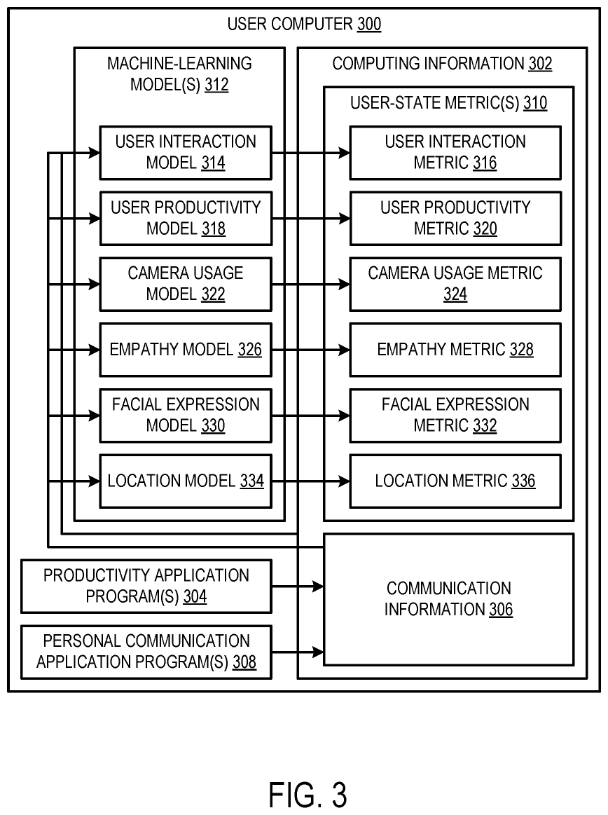

FIG. 3 shows an example user computer 300 configured to generate user-specific computing information 302 to assess a user's well being. For example, the user computer 300 may correspond to any of the plurality of user computers 204 shown in FIG. 2. Further, the computing information 302 may correspond to the computing information 210 shown in FIG. 2.

The user computer 300 may be configured to execute one or more productivity application programs 304 that are configured to generate computing information 302. The productivity application program(s) 304 may include any suitable type of application program that promotes user productivity. Non-limiting examples of productivity application programs 304 include word processing application programs, spreadsheet application programs, slide deck presentation application programs, note taking application programs, drawing/diagraming application programs, calendar application programs, and browser application programs. The computing information 302 generated by the productivity application program(s) 304 may indicate various aspects of user interactions that can inform an assessment of a user's well being. The computing information 302 generated by the productivity application program(s) 304 may take any suitable form. Non-limiting examples of such computing information may include the frequency at which different productivity application programs are used by the user, the computer/location from which the user uses different productivity application programs, other users that the user interacts with while using different productivity application programs, and language (written/typed or spoken) used in different productivity application programs.

In some examples, the computing information 302 generated by the productivity application program (s) 304 may include communication information 306. Non-limiting examples of communication information 306 that may be generated by the productivity application program(s) 304 include comments, audio segments, and video segments posted by a user to one or more other users in a document or file.

The user computer 300 may be configured to execute one or more personal communication application programs 308 that are configured to generate computing information 302. The personal communication application program(s) 308 may include any suitable type of application program that promotes user communication with other users. Non-limiting examples of personal communication application programs 308 include email application programs, messaging application programs, audio application programs, video application programs, audio/video conferencing application programs, and social network application programs. The computing information 302 generated by the personal communication application program(s) 308 may indicate various aspects of user interactions that can inform an assessment of a user's well being. The computing information 302 generated by the personal communication application program(s) 308 may take any suitable form. Non-limiting examples of such computing information may include the frequency at which different personal communication application programs are used by the user, the computer/location from which the user uses different personal communication application programs, other users that the user interacts with while using different personal communication application programs, language (written/typed or spoken) used in different personal communication application programs.

In some examples, the computing information 302 generated by the personal communication application program(s) 308 may include communication information 306. Non-limiting examples of communication information 306 that may be generate by the personal communication application program(s) 308 include email messages, text messages, audio transcripts, user audio segments, and user video segments.

The computing information 302 may be aggregated for a user over multiple different virtual interactions with different application programs and/or other users via the productivity application program(s) 304, the personal communication application program(s) 308, other application programs, an operating system, and/or computing services. Further, in some examples, application programs executing on the user computer 300 may be configured to obtain user-specific computing information 302 in other manners, such as explicitly requesting the computing information 302 from the user and/or inferring the computing information 302 based at least on user actions. The computing information 302 may be obtained for a user in any suitable manner.

In some implementations, the computing information 302 generated by the user computer 300 may include one or more user-state metrics 310 output from one or more machine-learning models 312 executing on the user computer 300. The machine-learning model(s) 312 may be previously-trained to quantify particular aspects of a user's well being in the form of the user-state metrics 310. The user-state metric(s) 310 indicate higher-level information that is distilled down from raw data collected by the user computer 300 and processed by the machine-learning model(s) 312. Such processing performed using the computing resources of the user computer 300 reduces a processing burden of the computing system 200 (shown in FIG. 2) relative to a configuration where a centralized computing system processes all the raw data unassisted. Further, such processing performed using the computing resources of the user computer 300 reduces an amount of information/data that is sent to the computing system 200 relative to a configuration where a centralized computing system processes all the raw data unassisted. These features provide the technical benefits of providing increased performance for the computing system 200 to generate well being assessments and reduced data transmission that equates to reduced power consumption and increases the amount of communication bandwidth available for other communications. In some implementations raw data may be sent from the user computer to a central computing system for remote processing; and in some implementations a combination of local and remote processing may be employed.

In one example, the machine learning model(s) 312 include a user interaction model 314 previously-trained to output a user interaction metric 316 indicating a level of user interaction based at least on the user communication information 306. For example, the user interaction metric 316 may track a frequency of communications (e.g., emails, messages, comments) from the user to other users, a frequency that the user attends and/or initiates scheduled interactions (e.g., via audio calls, video conferences), a frequency that the user is intentionally invited by other users to interact, and/or another suitable quantifier of a level of user interaction. The user interaction model 314 may determine the level of user interaction in any suitable manner. The level of interaction quantified by the user interaction metric 316 provides insight into a user's well being. For example, if a level of interaction of a user reduces in a statistically significant manner over a designated timeframe, then such behavior may indicate that the user's well being is decreasing.

In another example, the machine learning model(s) 312 include a user productivity model 318 previously-trained to output a user productivity metric 320 indicating a level of user productivity based at least on the computing information 302. A user's level of productivity may be determined based at least on a variety of factors including, but not limited to, a user input speed, a task completion time, a time taken for a user to take action responsive to a notification and/or to return to a previous task after taking action responsive to a notification. The user productivity model 318 may determine the level of user productivity in any suitable manner. The level of productivity quantified by the user productivity metric 320 provides insight into a user's well being. For example, if a level of productivity of a user reduces in a statistically significant manner over a designated timeframe, then such behavior may indicate that the user's well being is decreasing.

In another example, the machine learning model(s) 312 include a camera usage model 322 previously-trained to output a camera usage metric 324 indicating a level of camera usage during user interactions facilitated by the personal communication application program(s) 308. The camera usage model 322 may receive computing information 302 indicating each time a user's camera is turned on during a user interaction. Such camera usage may be reported by the personal communication application program(s) 308. In one example, the camera usage metric 324 may be represented as a scalar between 0-100, where 0 corresponds to a user not using the camera at all and 100 corresponding to a user using the camera during every user interaction. The camera usage model 322 may determine the level of camera usage in any suitable manner. The level of camera usage quantified by the camera usage metric 324 provides insight into a user's well being. For example, if a level of camera usage of a user reduces in a statistically significant manner over a designated timeframe, then such behavior may indicate that the user's well being is decreasing.

In another example, the machine learning model(s) 312 include an empathy model 326 previously-trained to output an empathy metric 328 indicating a level of user empathy based at least on user communication information 306 received from the personal communication application program(s) 308. In one example, the empathy model 326 may be previously-trained to recognize a representative cluster of text samples deemed to be anti-social or anti-collaborative. Further the empathy model 326 may compare the user's language in communications (e.g., determined from the communication information 306) with the representative cluster of text samples to determine the level of user empathy. In another example, the empathy model 326 may be previously-trained to analyze the user's voice patterns detected in user audio segments, such as during audio calls or video conferences (e.g., determined from the communication information 306) and determine the empathy metric 328 based at least on such analysis of the user's voice patterns. The user's voice patterns provide insight into the user's well being. For example, if the user's voice patterns include changes in pace, breaking down, repetition, long periods of silence, then such behavior may indicate that the user's well being is decreasing. The empathy model 326 may determine the level of user empathy in any suitable manner.

In another example, the machine learning model(s) 312 include a facial expression model 330 previously-trained to output a facial expression metric 332 indicating a user well being state based at least on images captured, via a camera, during user interactions facilitated by the personal communication application program(s) 308. In this example, the communication information 306 includes images that are provided as input to the facial expression model 330. In one example, the facial expression model 330 may be previously-trained to recognize different facial expressions and associate the different facial expressions with different emotions or well being states. The facial expression model 330 may analyze images of the user captured during video conferences and/or other video-based interactions and assess the user's well being state based at least on the user's facial expressions in the images. The user's facial expressions provide insight into the user's well being. For example, if the user's facial expressions change during a designated timeframe from positive facial expressions (e.g., smiling, laughing, engaged, and speaking) to neutral or negative facial expressions (e.g., frowning, checked out, not looking at the camera, not speaking, eyes closed for long periods), then such behavior may indicate that the user's well being is decreasing. The facial expression model 330 may assess the user's well being state based at least on any suitable analysis of images of the user.

In another example, the machine learning model(s) 312 include a location model 334 previously-trained to output a location metric 336 indicating a level to which a user's location changes on an interaction-to-interaction basis when interacting with the productivity application program(s) 304, the personal communication application program(s) 308, and/or any other application programs. In one example, the location model 334 may be configured to track a user's location based at least on logging IP addresses of computers when the user interacts with different application programs. The location model 334 may be configured to track the user's location in any suitable manner to generate the location metric 336. The level to which a user's location changes on an interaction-to-interaction basis provides insight into the user's well being. For example, if the user goes from working from different public locations (e.g., a restaurant or coffee shop) on a regular basis to working from the same private location (e.g., the user's mother's basement) during a designated timeframe, then such a change in behavior may indicate that the user's well being is decreasing. The location model 334 may determine the level to which a user's location changes on an interaction-to-interaction basis in any suitable manner.

The user computer 300 may be configured to execute any suitable number of different machine-learning models to output any suitable user-state metric that can be used to assess a user's well being. In some examples, one or more of the machine-learning modes may be previously-trained neural networks. The computing information 302 generated by the user computer 300 may be sent to the computing system 200 (shown in FIG. 2), so that the computing system 200 can use the computing information 302 to assess a user's well being. In some examples, the computing information 302 is sent from the productivity application program(s) 304 to the computing system 200. In some examples, the computing information 302 is sent from the personal communication application program(s) 308 to the computing system 200. In some examples, the computing information is sent from both the productivity application program(s) 304 and the personal communication application program(s) 308 to the computing system 200. While machine learning models can advantageously diagnose and summarize complicated user behavior patterns, in some implementations the hard-coded heuristics or other assessment logic may be used in addition to or instead of machine learning models.

The computing information 302 generated by the user computer 300 is representative of computing information that may be generated by any user computer of a plurality of user computers. In some examples, computing information generated for a user by a plurality of different user computers may be sent to the computing system 200 (shown in FIG. 2) to assess the user's well being.

Returning to FIG. 2, the computing system 200 includes a well being assessment machine 220 configured to generate a user's well being score 222 based at least on computing information 218 attributed to the user account 214 of the user. The well being assessment machine 220 may be configured to generate the user's well being score 222 in any suitable manner. In one example, the well being assessment machine 220 may include a previously trained machine-learning model, such as a neural network. In particular, the model may be previously-trained to receive one or more of the plurality of user-state metrics 310 (shown in FIG. 3) and optionally any other user-specific parameters derived from the computing information 302 as input, and output the user's well being score 222 based at least on the user-state metric(s) and the other user-specific parameters, if the other user-specific parameters are provided as input.

The user state metrics and user's well being score 222 may take any suitable form. In some examples, the user state metrics and/or user's well being score may include a number (e.g., an integer/scalar). In other examples, the user state metrics and/or user's well being score 222 may include a multi-dimensional score (e.g., represented as a vector with a plurality of coefficients relating to different aspects of a user's well being). In general, the user's well being score 222 may be represented using any suitable data structure(s) for which a rate of change of the user's well being can be assessed. For example, in the case of a user well being score that is represented by an integer variable, the well being may be represented over time as a function of this variable, and the change in well being may be represented by the first derivative of this function and/or the net change in this value over a certain period of time. As another example, in the case of a user well being score that is represented by a multidimensional vector, a change in well being may be calculated as a geometric distance between such vectors at different times. These are just examples, and other mechanisms for representing the score and/or calculating a rate of change of the score may be used.

The well being assessment machine 220 is configured to progressively update the well being score 222 over time for the user account 214 based at least on the computing information 218. In other words, as the computing information 218 attributed to the user account 214 is updated over time, the well being assessment machine 220 may update the well being score 222 based at least on the updated computing information. The user's well being score 222 may be progressively updated over time in order to observe and track changes in the user's well being. The well being assessment machine 220 may update the user's well being score 222 according to any suitable frequency and/or any suitable timeframe that allows for such observation and tracking of changes in the user's well being.

The computing system 200 includes a mitigation machine 224 configured to track a rate of change of the user's well being score 222 over time. In particular, the mitigation machine 224 is configured to detect when the user's well being score 222 decreases and compares a rate of decrease 226 of the user's well being score 222 to a threshold rate of decrease 228. Further, the mitigation machine 224 is configured to perform one or more mitigation operations 230 associated with the user account 214 based at least on an above threshold 228 rate of decrease 226 of the user's well being score 222. The rate of decrease 226 may be measured over any suitable timeframe.

By using the threshold rate of decrease of the well being score as the trigger for performing the mitigation operations, a distinct change in the user's behavior can be accurately identified before intervening and providing mitigation to improve the user's well being. Such an approach may be more accurate than acting on general assessments or snapshot assessments of a user's well being. In some implementations, different mitigations may be triggered responsive to different thresholds being exceeded.

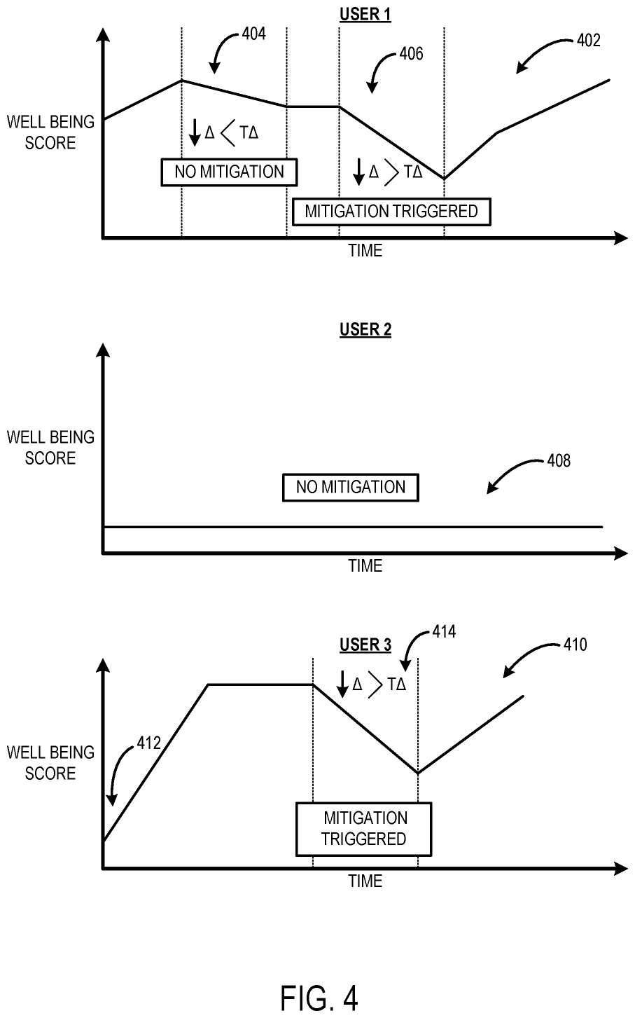

FIG. 4 shows a plurality of example scenarios in which users having different well being scores are handled differently by the mitigation machine 224 based at least on different rates of change of the well being scores. In each of the plurality of scenarios, a user's well being score is tracked over time. In these examples, the user well being is represented by a single number, graphed on the vertical axis; and the rate of change is represented by the slope of the line. However, other scoring systems may be used to assess well being and the rate of change. At 402, in a first scenario, a first user's well being score fluctuates up and down over time. At 404, in a first timeframe, the first user's well being score decreases (negative slope), and in this case the rate of decrease is less than the threshold rate of decrease, so mitigation operations are triggered. At 406, in a second timeframe, the first user's well being score again decreases, and in this case the rate of decrease is greater than the threshold rate of decrease (i.e., the negative slope exceeds the threshold for a given period). This triggers the mitigation machine to perform one or more mitigation operations to improve the first user's well being score.

At 408, in a second scenario, a second user's well being score is generally low and does not fluctuate over time. Although the second user's well being score is low, the well being score is consistent over time indicating that the second user's behavior is consistent. In this case, the mitigation machine does not perform any mitigation operations, because there is no detected change in the second user's behavior that would indicate a decrease in the second user's well being.

At 410, in a third scenario, a third user's well being score fluctuates sharply up and down over time. Initially, at 412, the third user's well being score is very low and increases sharply becoming very high. Then, in a timeframe 414, the third user's well being score decreases sharply, such that the rate of decrease is greater than the threshold rate of decrease. This triggers the mitigation machine to perform one or more mitigation operations to improve the third user's well being score. In this scenario, even though the third user's well being score in the timeframe 412 is higher than the initial well being score at 412, mitigation operation(s) are still performed with the goal being to improve the third user's well being to counteract negative changes in the third user's behavior.

Returning to FIG. 2, the mitigation machine 224 may be configured to perform any suitable mitigation operation(s) 230 to improve a user's well being responsive to an above threshold rate of decrease of the user's well being score. A mitigation operation may provide proactive actionable steps performed by computer application program(s) (or service(s), operating system) executing on one or more of the computing system 200 and/or any of the plurality of user computers 204 that prompt the user to improve the user's well being.

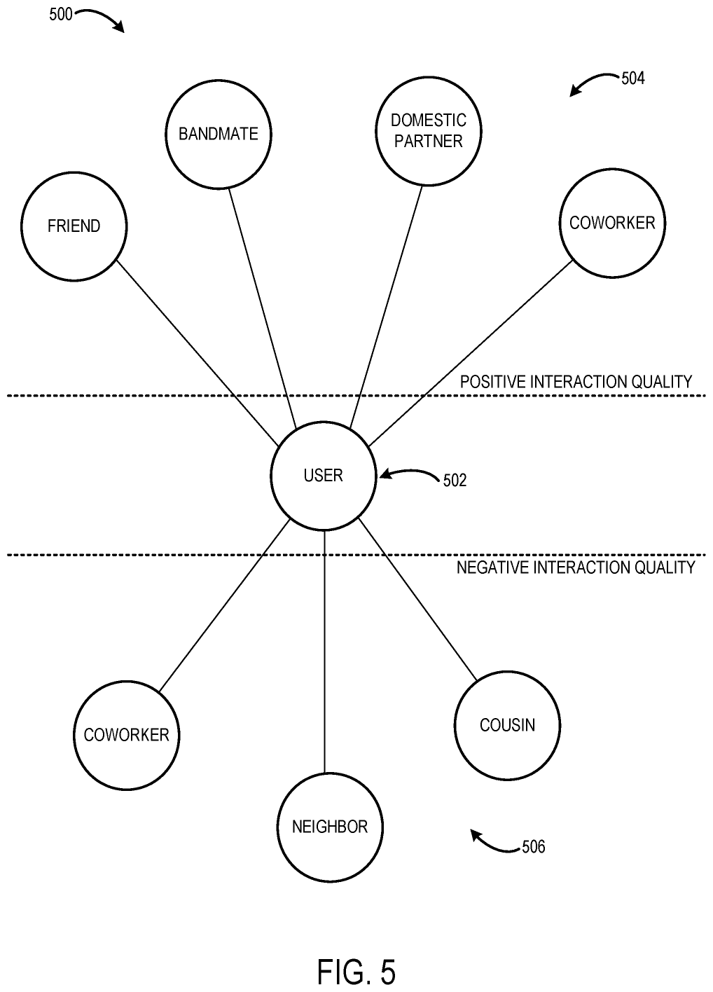

In some implementations, the mitigation operation 230 may include actionable steps that prompt a user to connect with other people in order to improve the user's well being. In one example, the user account 214 includes a social network graph 500 (shown in FIG. 5) including a plurality of different users having different interpersonal connections/relationships with the user 502. The mitigation machine 224 is configured to, for each different user in the social network graph 500, determine an interaction quality indicating a level to which the different user influences the well being state of the user 502 during user interactions. For example, such influence may be observed by changes in the user's well being score 222 during or subsequent to such user interactions. The mitigation machine 224 may determine the interaction quality in any suitable manner. In some examples, the interaction quality may be determined based at least on quality and quantity of interactions. In some examples, the interaction quality may be determined based at least on one or more of duration of interactions, frequency of interactions, language used during interactions, voice patterns of the user, facial expressions of the user during interactions. In some examples, the interaction quality may be determined based at least on the user-state metric(s) 310 shown in FIG. 3 and any other suitable computing information 302 attributed to the user. In some implementations, a user's well being score and/or changes to the user's well being score may be tracked before, during, and after a user interacts with different people, and such assessments may be associated with the different people in the user's social network graph.

In the illustrated example, in the social network graph 500, a first plurality of users 504 have a positive interaction quality with the user 502 meaning that the first plurality of users 504 have a positive influence on the well being of the user 502. In this example, the users having a positive interaction quality include a friend, a bandmate, a domestic partner, and a first coworker. Further, a second plurality of users have a negative interaction quality with the user 502 meaning that the second plurality of users 504 have a negative influence on the well being of the user 502. In this example, the user having a negative interaction quality include a second coworker, a neighbor, and a cousin.

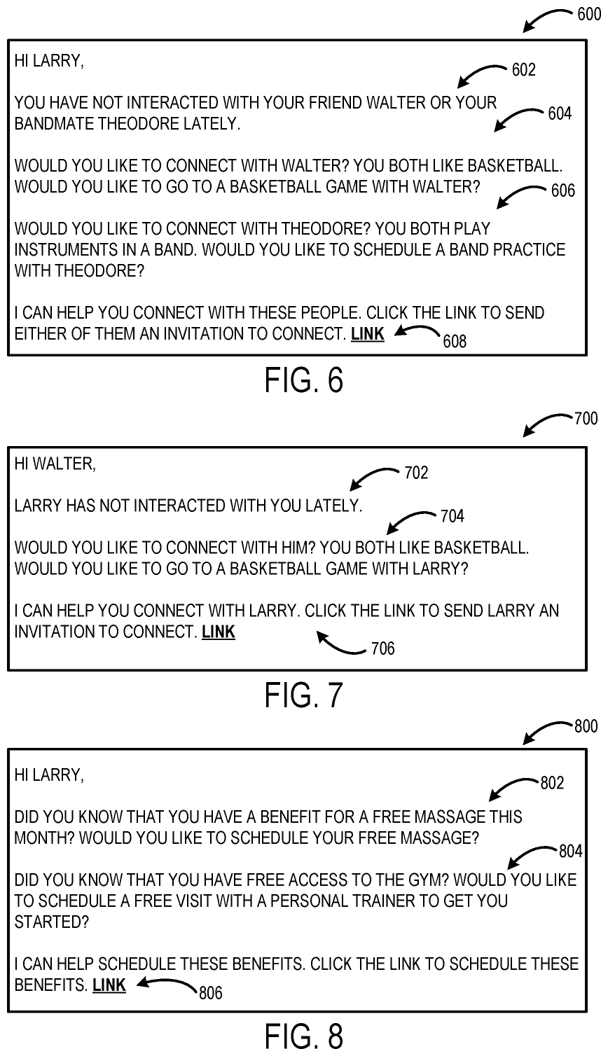

The mitigation machine 224 may be configured to perform mitigation operation(s) 230 that encourage the user 502 to interact with other users 504 in the social network graph 500 having a positive interaction quality. In one example, the mitigation machine 224 is configured to perform a mitigation operation that includes sending, to the user computer 204A via the network communication subsystem 202, an invitation notification recommending user interaction with different users in the social network graph 500 (shown in FIG. 5) that have a positive interaction quality. FIG. 6 shows an example invitation notification 600 that may be sent to a user named ‘Larry’ as a mitigation operation that is triggered based at least on an above threshold decrease of Larry's well being score. The invitation notification 600 recommends user interaction with different users in Larry's social network graph that have a positive interaction quality when interacting with Larry. The invitation notification 600 includes a reminder 602 that Larry has not interacted with other users in his social network graph lately. In particular, the reminder 602 specifies that Larry has not interacted with his friend ‘Walter’ or his bandmate ‘Theodore.’ The invitation notification 600 further includes recommendations 604, 606 of activities corresponding to common interests between Larry and each of the other users to encourage user interaction. A first recommendation 604 suggests that Larry go to a basketball game with Walter, because they both like basketball. A second recommendation 606 suggests that Larry schedule a band practice with Theodore, because they both play instruments in a band. For example, the mitigation machine 224 (shown in FIG. 2) may be configured to populate the recommendations 604 with computing information attributed to user accounts 216 (shown in FIG. 2) of the users in the social network graph 500 (shown in FIG. 5). The invitation notification 600 includes a scheduling prompt 608 that is selectable via user input to automatically generate invitations to the other users to interface with Larry. For example, the invitations may be generated as emails or calendar invites. The invitation notification 600 presents proactive steps that the user can take to improve the user's well being score.

In another example, the mitigation machine 224 is configured to perform a mitigation operation that includes sending, via the network communication subsystem 202, to a different user computer 204B corresponding to a different user in the social network graph 500 (shown in FIG. 5) that has a positive interaction quality, a wellness check notification recommending the different user initiate user interaction. FIG. 7 shows an example wellness check notification 700 that may be sent to Larry's friend Walter as a mitigation operation that is triggered based at least on an above threshold decrease of Larry's well being score. The wellness check notification 700 includes a reminder 702 that Walter has not interacted with Larry lately. The wellness check notification 700 further includes a recommendation 704 of an activity corresponding to a common interest between Walter and Larry. The recommendation 704 suggests that Walter go to a basketball game with Larry, because they both like basketball. The wellness check notification 700 includes a scheduling prompt 706 that is selectable via user input to automatically generate an invitation to Larry to interface with Walter. For example, the invitation may be generated as an email or a calendar invite.

In another example, the mitigation machine 224 is configured to perform a mitigation operation that includes sending, to the user computer 204A via the network communication subsystem 202, an invitation notification recommending user interaction with different users that are tracked by the computing system 200 and are not in the user's social network graph. In other words, the mitigation machine 224 may recommend user interaction with “new” users that have not previously had robust user interactions with the user. The mitigation machine 224 may recommend new users based at least on any suitable similarities or common parameters. For example, mitigation machine 224 may recommend new users based at least on having a common “age” and “country of residence”. As another example, the mitigation machine 224 may recommend new users based at least on the user and the new users having common interests. In some examples, the mitigation machine 224 may be configured to predict the quality of interaction of the new users with the user based at least on the interaction quality with other users. If a new user is predicted to be a good fit in terms of interaction but is estimated to have a negative interaction quality, then the new user is not recommended for user interaction. In some examples, the mitigation machine 224 may be configured to automatically schedule a meeting (e.g., a video conference call) with the recommended new people to socialize.

In another example, the mitigation machine 224 is configured to perform a mitigation operation that includes at least temporarily suppressing notifications from different users in the social network graph 500 (shown in FIG. 5) that have a negative interaction quality. Referring back to the example of Larry, Larry's coworker, neighbor, and cousin all have negative interaction qualities, so notifications sent from these users are at least temporarily suppressed by the mitigation machine 224. For example, emails, text messages, social network posts, and other types of notifications may be suppressed. Such notifications may be suppressed for any suitable duration. In some examples, the notifications may be delayed from being presented to the user until the user's well being score has changed (e.g., trending upward). In other examples, the notifications may be delayed from being presented to the user for a designated delay-period. The mitigation machine 224 may be configured to suppress or delay notifications in any suitable manner. Suppressing such notification reduces the likelihood that the users' well being score decreases further and allows the user to have positive interactions that facilitate improvements in the user's well being. In some implementations, natural language processing may be used to assess whether it is safe to at least temporarily suppress such communication, so as to avoid suppressing urgent and/or important communications.

In another example, the mitigation machine 224 is configured to perform a mitigation operation that includes sending, to the user computer 204A via the network communication subsystem 202, a benefits reminder notification indicating well being state improving benefits that are currently available for the user account 214. FIG. 8 shows an example benefits reminder notification 800 that may be sent to Larry as a mitigation operation that is triggered based at least on an above threshold decrease of Larry's well being score. The benefits reminder notification 800 includes a first benefits reminder 802 indicating that Larry has a benefit for a free message (e.g., as part of an employee benefits package). The benefits reminder notification 800 includes a second reminder indicating that Larry has free access to a gym and a free visit with a personal trainer. The benefits reminder notification 800 includes a scheduling prompt 806 that is selectable via user input to automatically schedule times to user the free benefits. The benefits reminder notification 800 presents proactive steps that the user can take to improve the user's well being score.

The notifications 600, 700, 800 shown in FIGS. 6-8 may take any suitable form. In some examples, the notifications may be emails. In some examples, the notifications may be text messages. In some examples, the notifications may be user interface (UI) messages. In some examples, the notifications may be presented by an AI-based automated assistant trained to learn how a user prefers such notifications and which types of notifications, and the timing at which they are delivered, have the largest positive effect on the user's well being score.

The mitigation machine 224 may be configured to perform any suitable mitigation operations to improve a user's well being score. The mitigation machine 224 may be configured to perform any suitable number of mitigation operations at any suitable frequency. In some examples, the frequency and/or type of mitigation operation that is performed may be based at least on both the raw well being score and the rate of change of the well being score.

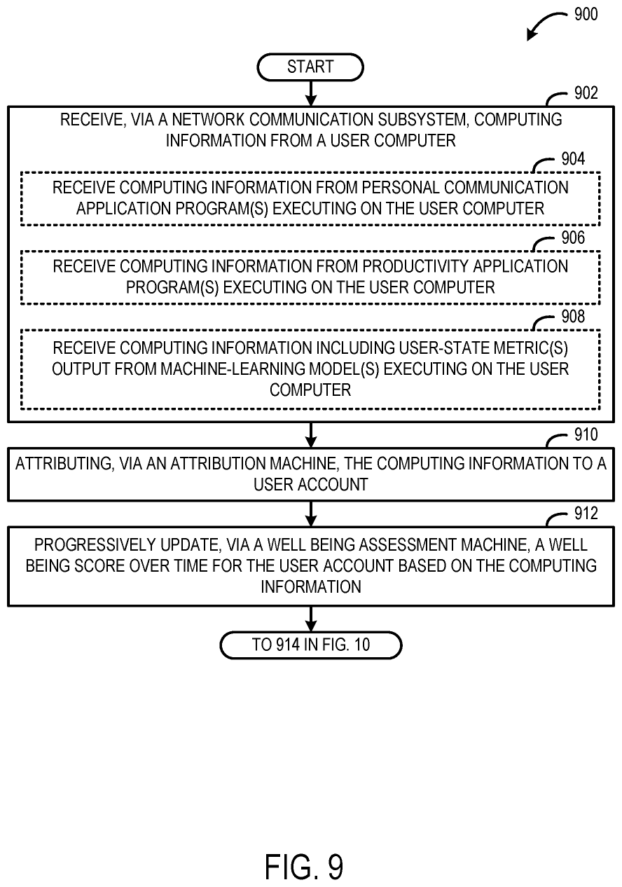

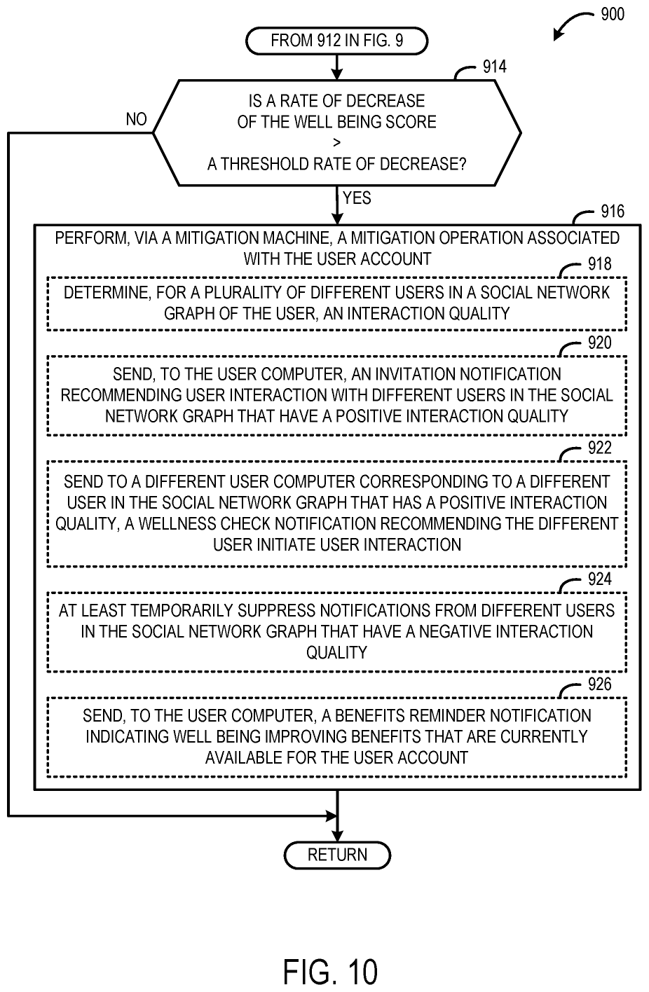

FIGS. 9-10 show an example computer-implemented method 900 for assessing a user's well being. For example, the method 900 may be performed by the computing system 200 shown in FIG. 2. In some examples, at least some of the method 900 may be at least partially performed by one or more of the plurality of user computers 204.

In FIG. 9, at 902, the method 900 includes receiving, via a network communication subsystem, computing information from a user computer. In some implementations, at 904, the method 900 may include receiving computing information from one or more personal communication application programs executing on the user computer. In some implementations, at 906, the method 900 may include receiving computing information from one or more productivity application programs executing on the user computer. In some implementations, at 908, the method 900 may include receiving computing information including one or more user-state metrics output from one or more machine-learning models executing on the user computer.

At 910, the method 900 includes attributing, via an attribution machine, the computing information to a user account. At 912, the method 900 includes progressively updating, via a well being assessment machine, a well being score over time for the user account based at least on the computing information.

In FIG. 10, at 914, the method 900 includes determining whether a rate of decrease of the well being score is greater than a threshold rate of decrease. If the rate of decrease of the well being score is greater than the threshold rate of decrease, then the method 900 moves to 916. Otherwise, the rate of decrease of the well being score is not greater than the threshold rate of decrease and the method 900 returns to other operations.

At 916, the method 900 includes performing, via a mitigation machine, a mitigation operation associated with the user account based at least on an above threshold rate of decrease of the well being score. In some implementations, at 918, the method 900 may include determining, for a plurality of different users in a social network graph of the user, an interaction quality. The interaction quality indicates a level to which the different user influences the user well being state during user interactions. In some implementations, at 920, the method 900 may include sending, to the user computer via the network communication subsystem, an invitation notification recommending user interaction with different users in the social network graph that have a positive interaction quality. In some implementations, at 922, the method 900 may include sending, via the network communication subsystem, to a different user computer corresponding to a different user in the social network graph that has a positive interaction quality, a wellness check notification recommending the different user initiate user interaction. In some implementations, at 924, the method 900 may include at least temporarily suppressing notifications from different users in the social network graph that have a negative interaction quality. In some implementations, at 926, the method 900 may include sending, to the user computer via the network communication subsystem, a benefits reminder notification indicating well being state improving benefits that are currently available for the user account.

The computer-implemented user health assessment method may be performed to quantitatively assess a user's well being based on computing information that is collected based at least on computer-based user interactions. Further, computer-based mitigation operations are performed based at least on such well being assessments in order to proactively improve a user's well being. For example, such mitigation operations may include actionable steps that prompt a user to connect with other people or prompt other people to reach out and connect with the user. In particular, mitigation operation(s) are triggered based at least on an above threshold rate of decrease in the user's well being score. By using the rate of decrease of the well being score as the trigger for performing the mitigation operations, a distinct change in the user's behavior can be accurately identified before intervening and providing mitigation to improve the user's well being. Such an approach may be more accurate than acting on general or snapshot assessments of a user's well being.

The methods and processes described herein may be tied to a computing system of one or more computing devices. In particular, such methods and processes may be implemented as an executable computer-application program, a network-accessible computing service, an application-programming interface (API), a library, or a combination of the above and/or other compute resources.

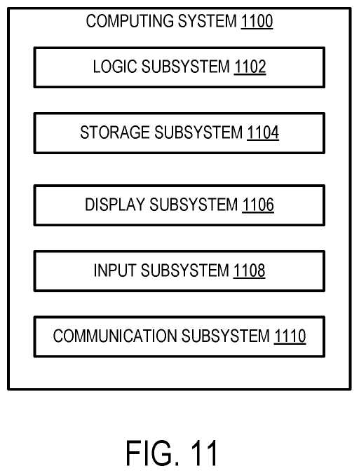

FIG. 11 schematically shows a simplified representation of a computing system 1100 configured to provide any to all of the compute functionality described herein. For example, the computing system 1100 may correspond to the user computer 102 shown in FIG. 1, the computing system 200 and the plurality of user computers 204 shown in FIG. 2, and the user computer 300 shown in FIG. 3. Computing system 1100 may take the form of one or more personal computers, network-accessible server computers, tablet computers, home-entertainment computers, gaming devices, mobile computing devices, mobile communication devices (e.g., smart phone), virtual/augmented/mixed reality computing devices, wearable computing devices, Internet of Things (IoT) devices, embedded computing devices, and/or other computing devices.

Computing system 1100 includes a logic subsystem 1102 and a storage subsystem 1104. Computing system 1100 may optionally include a display subsystem 1106, input subsystem 1108, communication subsystem 1110, and/or other subsystems not shown in FIG. 11.

Logic subsystem 1102 includes one or more physical devices configured to execute instructions. For example, the logic subsystem may be configured to execute instructions that are part of one or more applications, services, or other logical constructs. The logic subsystem may include one or more hardware processors configured to execute software instructions. Additionally or alternatively, the logic subsystem may include one or more hardware or firmware devices configured to execute hardware or firmware instructions. Processors of the logic subsystem may be single-core or multi-core, and the instructions executed thereon may be configured for sequential, parallel, and/or distributed processing. Individual components of the logic subsystem optionally may be distributed among two or more separate devices, which may be remotely located and/or configured for coordinated processing. Aspects of the logic subsystem may be virtualized and executed by remotely-accessible, networked computing devices configured in a cloud-computing configuration.

Storage subsystem 1104 includes one or more physical devices configured to temporarily and/or permanently hold computer information such as data and instructions executable by the logic subsystem. When the storage subsystem includes two or more devices, the devices may be collocated and/or remotely located. Storage subsystem 1104 may include volatile, nonvolatile, dynamic, static, read/write, read-only, random-access, sequential-access, location-addressable, file-addressable, and/or content-addressable devices. Storage subsystem 1104 may include removable and/or built-in devices. When the logic subsystem executes instructions, the state of storage subsystem 1104 may be transformed—e.g., to hold different data.

Aspects of logic subsystem 1102 and storage subsystem 1104 may be integrated together into one or more hardware-logic components. Such hardware-logic components may include program- and application-specific integrated circuits (PASIC/ASICs), program- and application-specific standard products (PSSP/ASSPs), system-on-a-chip (SOC), and complex programmable logic devices (CPLDs), for example.

The logic subsystem and the storage subsystem may cooperate to instantiate one or more logic machines. As used herein, the terms “machine” (e.g., attribution machine, well being assessment machine, and mitigation machine) and machine learning model (e.g., user interaction model, user productivity model, camera usage model, empathy model, facial expression model, and location model) are used to collectively refer to the combination of hardware, firmware, software, instructions, and/or any other components cooperating to provide computer functionality. In other words, “machines” and “models” are never abstract ideas and always have a tangible form. A machine and/or model may be instantiated by a single computing device, or a machine may include two or more sub-components instantiated by two or more different computing devices. In some implementations a machine includes a local component (e.g., software application executed by a computer processor) cooperating with a remote component (e.g., cloud computing service provided by a network of server computers). The software and/or other instructions that give a particular machine its functionality may optionally be saved as one or more unexecuted modules on one or more suitable storage devices.

Machines may be implemented using any suitable combination of state-of-the-art and/or future machine learning (ML), artificial intelligence (AI), and/or natural language processing (NLP) techniques. Non-limiting examples of techniques that may be incorporated in an implementation of one or more machines include support vector machines, multi-layer neural networks, convolutional neural networks (e.g., including spatial convolutional networks for processing images and/or videos, temporal convolutional neural networks for processing audio signals and/or natural language sentences, and/or any other suitable convolutional neural networks configured to convolve and pool features across one or more temporal and/or spatial dimensions), recurrent neural networks (e.g., long short-term memory networks), Transformer-based machine learning models (e.g., Bidirectional Encoder Representations from Transformers), associative memories (e.g., lookup tables, hash tables, Bloom Filters, Neural Turing Machine and/or Neural Random Access Memory), word embedding models (e.g., GloVe or Word2Vec), unsupervised spatial and/or clustering methods (e.g., nearest neighbor algorithms, topological data analysis, and/or k-means clustering), graphical models (e.g., (hidden) Markov models, Markov random fields, (hidden) conditional random fields, and/or AI knowledge bases), and/or natural language processing techniques (e.g., tokenization, stemming, constituency and/or dependency parsing, and/or intent recognition, segmental models, and/or super-segmental models (e.g., hidden dynamic models)).

In some examples, the methods and processes described herein may be implemented using one or more differentiable functions, wherein a gradient of the differentiable functions may be calculated and/or estimated with regard to inputs and/or outputs of the differentiable functions (e.g., with regard to training data, and/or with regard to an objective function). Such methods and processes may be at least partially determined by a set of trainable parameters. Accordingly, the trainable parameters for a particular method or process may be adjusted through any suitable training procedure, in order to continually improve functioning of the method or process.

Non-limiting examples of training procedures for adjusting trainable parameters include supervised training (e.g., using gradient descent or any other suitable optimization method), zero-shot, few-shot, unsupervised learning methods (e.g., classification based at least on classes derived from unsupervised clustering methods), reinforcement learning (e.g., deep Q learning based at least on feedback) and/or generative adversarial neural network training methods, belief propagation, RANSAC (random sample consensus), contextual bandit methods, maximum likelihood methods, and/or expectation maximization. In some examples, a plurality of methods, processes, and/or components of systems described herein may be trained simultaneously with regard to an objective function measuring performance of collective functioning of the plurality of components (e.g., with regard to reinforcement feedback and/or with regard to labelled training data). Simultaneously training the plurality of methods, processes, and/or components may improve such collective functioning. In some examples, one or more methods, processes, and/or components may be trained independently of other components (e.g., offline training on historical data).

Language models may utilize vocabulary features to guide sampling/searching for words for recognition of speech. For example, a language model may be at least partially defined by a statistical distribution of words or other vocabulary features. For example, a language model may be defined by a statistical distribution of n-grams, defining transition probabilities between candidate words according to vocabulary statistics. The language model may be further based at least on any other appropriate statistical features, and/or results of processing the statistical features with one or more machine learning and/or statistical algorithms (e.g., confidence values resulting from such processing). In some examples, a statistical model may constrain what words may be recognized for an audio signal, e.g., based at least on an assumption that words in the audio signal come from a particular vocabulary.

Alternately or additionally, the language model may be based at least on one or more neural networks previously trained to represent audio inputs and words in a shared latent space, e.g., a vector space learned by one or more audio and/or word models (e.g., wav2letter and/or word2vec). Accordingly, finding a candidate word may include searching the shared latent space based at least on a vector encoded by the audio model for an audio input, in order to find a candidate word vector for decoding with the word model. The shared latent space may be utilized to assess, for one or more candidate words, a confidence that the candidate word is featured in the speech audio.

The language model may be used in conjunction with an acoustical model configured to assess, for a candidate word and an audio signal, a confidence that the candidate word is included in speech audio in the audio signal based at least on acoustical features of the word (e.g., mel-frequency cepstral coefficients, formants, etc.). Optionally, in some examples, the language model may incorporate the acoustical model (e.g., assessment and/or training of the language model may be based at least on the acoustical model). The acoustical model defines a mapping between acoustic signals and basic sound units such as phonemes, e.g., based at least on labelled speech audio. The acoustical model may be based at least on any suitable combination of state-of-the-art or future machine learning (ML) and/or artificial intelligence (AI) models, for example: deep neural networks (e.g., long short-term memory, temporal convolutional neural network, restricted Boltzmann machine, deep belief network), hidden Markov models (HMM), conditional random fields (CRF) and/or Markov random fields, Gaussian mixture models, and/or other graphical models (e.g., deep Bayesian network). Audio signals to be processed with the acoustic model may be pre-processed in any suitable manner, e.g., encoding at any suitable sampling rate, Fourier transform, band-pass filters, etc. The acoustical model may be trained to recognize the mapping between acoustic signals and sound units based at least on training with labelled audio data. For example, the acoustical model may be trained based at least on labelled audio data comprising speech audio and corrected text, in order to learn the mapping between the speech audio signals and sound units denoted by the corrected text. Accordingly, the acoustical model may be continually improved to improve its utility for correctly recognizing speech audio.

In some examples, in addition to statistical models, neural networks, and/or acoustical models, the language model may incorporate any suitable graphical model, e.g., a hidden Markov model (HMM) or a conditional random field (CRF). The graphical model may utilize statistical features (e.g., transition probabilities) and/or confidence values to determine a probability of recognizing a word, given the speech audio and/or other words recognized so far. Accordingly, the graphical model may utilize the statistical features, previously trained machine learning models, and/or acoustical models to define transition probabilities between states represented in the graphical model.

When included, display subsystem 1106 may be used to present a visual representation of data held by storage subsystem 1104. This visual representation may take the form of a graphical user interface (GUI). Display subsystem 1106 may include one or more display devices utilizing virtually any type of technology. In some implementations, display subsystem may include one or more virtual-, augmented-, or mixed reality displays.

When included, input subsystem 1108 may comprise or interface with one or more input devices. An input device may include a sensor device or a user input device. Examples of user input devices include a keyboard, mouse, touch screen, or game controller. In some embodiments, the input subsystem may comprise or interface with selected natural user input (NUI) componentry. Such componentry may be integrated or peripheral, and the transduction and/or processing of input actions may be handled on- or off-board. Example NUI componentry may include a microphone for speech and/or voice recognition; an infrared, color, stereoscopic, and/or depth camera for machine vision and/or gesture recognition; a head tracker, eye tracker, accelerometer, and/or gyroscope for motion detection and/or intent recognition.

When included, communication subsystem 1110 may be configured to communicatively couple computing system 1100 with one or more other computing devices. Communication subsystem 1110 may include wired and/or wireless communication devices compatible with one or more different communication protocols. The communication subsystem may be configured for communication via personal-, local- and/or wide-area networks.

When the methods and processes described herein incorporate ML and/or AI components, the ML and/or AI components may make decisions based at least partially on training of the components with regard to training data. Accordingly, the ML and/or AI components can and should be trained on diverse, representative datasets that include sufficient relevant data for diverse users and/or populations of users. In particular, training data sets should be inclusive with regard to different human individuals and groups, so that as ML and/or AI components are trained, their performance is improved with regard to the user experience of the users and/or populations of users.

ML and/or AI components may additionally be trained to make decisions so as to minimize potential bias towards human individuals and/or groups. For example, when AI systems are used to assess any qualitative and/or quantitative information about human individuals or groups, they may be trained so as to be invariant to differences between the individuals or groups that are not intended to be measured by the qualitative and/or quantitative assessment, e.g., so that any decisions are not influenced in an unintended fashion by differences among individuals and groups.

ML and/or AI components may be designed to provide context as to how they operate, so that implementers of ML and/or AI systems can be accountable for decisions/assessments made by the systems. For example, ML and/or AI systems may be configured for replicable behavior, e.g., when they make pseudo-random decisions, random seeds may be used and recorded to enable replicating the decisions later. As another example, data used for training and/or testing ML and/or AI systems may be curated and maintained to facilitate future investigation of the behavior of the ML and/or AI systems with regard to the data. Furthermore, ML and/or AI systems may be continually monitored to identify potential bias, errors, and/or unintended outcomes.

This disclosure is presented by way of example and with reference to the associated drawing figures. Components, process steps, and other elements that may be substantially the same in one or more of the figures are identified coordinately and are described with minimal repetition. It will be noted, however, that elements identified coordinately may also differ to some degree. It will be further noted that some figures may be schematic and not drawn to scale. The various drawing scales, aspect ratios, and numbers of components shown in the figures may be purposely distorted to make certain features or relationships easier to see.

In an example, a computing system comprises a network communication subsystem configured to communicate with a plurality of user computers, an attribution machine configured to attribute, to a user account, computing information that the network communication subsystem receives from a user computer of the plurality of user computers, a well being assessment machine configured to progressively update a well being score over time for the user account based at least on the computing information, and a mitigation machine configured to perform a mitigation operation associated with the user account based at least on an above threshold rate of decrease of the well being score. In this example and/or other examples, the computing information attributed to the user account may include one or more user-state metrics output from one or more machine-learning models executing on the user computer from which the computing information is received. In this example and/or other examples, the one or more user-state metrics may include a user interaction metric indicating a level of user interaction based at least on user communication information generated by one or more productivity application programs and/or one or more personal communication application programs. In this example and/or other examples, the one or more user-state metrics may include a user productivity metric indicating a level of user productivity based at least on computing information generated by one or more productivity application programs. In this example and/or other examples, the one or more user-state metrics may include a camera usage metric indicating a level of camera usage during user interactions facilitated by one or more personal communication application programs. In this example and/or other examples, the one or more user-state metrics may include an empathy metric indicating a level of user empathy based at least on user communication information received from one or more personal communication application programs. In this example and/or other examples, the communication information may include one or more email messages, text messages, audio transcripts, and/or comments posted in communication application programs and/or productivity application programs. In this example and/or other examples, the communication information may include a user audio segment, and wherein the empathy metric is determined based at least on voice patterns detected in the user audio segment. In this example and/or other examples, the one or more user-state metrics may include a facial expression metric indicating a user well being state based at least on images captured, via a camera, during user interactions facilitated by one or more personal communication application programs. In this example and/or other examples, the one or more user-state metrics may include a location metric indicating a level to which a user's location changes on an interaction-to-interaction basis when interacting with one or more of a personal communication application program and a productivity application program. In this example and/or other examples, computing information attributed to the user account may be received from one or more personal communication application programs and one or more productivity application programs. In this example and/or other examples, the user account may include a social network graph including a plurality of different users, the mitigation machine may be configured to, for each different user in the social network graph, determine an interaction quality indicating a level to which the different user influences the user well being state during user interactions, and the mitigation operation may include sending, to the user computer via the network communication subsystem, an invitation notification recommending user interaction with a different user in the social network graph that has a positive interaction quality. In this example and/or other examples, the mitigation operation may include sending, via the network communication subsystem, to a different user computer corresponding to the different user in the social network graph that has a positive interaction quality, a wellness check notification recommending the different user initiate user interaction. In this example and/or other examples, the mitigation operation may include at least temporarily suppressing notifications from a different user in the social network graph that has a negative interaction quality. In this example and/or other examples, the mitigation operation may include sending, to the user computer via the network communication subsystem, a benefits reminder notification indicating well being state improving benefits that are currently available for the user account.

In another example, a computer-implemented method comprises receiving, via a network communication subsystem, computing information from a user computer, attributing, via an attribution machine, the computing information to a user account, progressively updating, via a well being assessment machine, a well being score over time for the user account based at least on the computing information; and performing, via a mitigation machine, a mitigation operation associated with the user account based at least on an above threshold rate of decrease of the well being score. In this example and/or other examples, the user account may include a social network graph including a plurality of different users, the mitigation machine may be configured to, for each different user in the social network graph, determine an interaction quality indicating a level to which the different user influences the user well being state during user interactions, and the mitigation operation may include sending, to the user computer via the network communication subsystem, an invitation notification recommending user interaction with a different user in the social network graph that has a positive interaction quality. In this example and/or other examples, the mitigation operation may include sending, via the network communication subsystem, to a different user computer corresponding to the different user in the social network graph that has a positive interaction quality, a wellness check notification recommending the different user initiate user interaction. In this example and/or other examples, the mitigation operation may include at least temporarily suppressing notifications from a different user in the social network graph that has a negative interaction quality.

In yet another example, a computing system comprises a network communication subsystem configured to communicate with a plurality of user computers, an attribution machine configured to attribute, to a user account, computing information that the network communication subsystem receives from a user computer of the plurality of user computers, the computing information including one or more user-state metrics output from one or more machine-learning models executing on the user computer from which the computing information is received, a well being assessment machine configured to progressively update a well being score over time for the user account based at least on the user-state metrics, and a mitigation machine configured to perform a mitigation operation associated with the user account based at least on an above threshold rate of decrease of the well being score.

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. A computing system, comprising:
a network communication subsystem configured to communicate with a plurality of user computers;
an attribution machine configured to attribute, to a user account, computing information that the network communication subsystem receives from a user computer of the plurality of user computers;
a well being assessment machine configured to progressively update a well being score over time for the user account based at least on the computing information; and
a mitigation machine configured to perform a mitigation operation associated with the user account based at least on an above threshold rate of decrease of the well being score.

2. The computing system of claim 1, wherein the computing information attributed to the user account includes one or more user-state metrics output from one or more machine-learning models executing on the user computer from which the computing information is received.

3. The computing system of claim 2, wherein the one or more user-state metrics includes a user interaction metric indicating a level of user interaction based at least on user communication information generated by one or more productivity application programs and/or one or more personal communication application programs.

4. The computing system of claim 2, wherein the one or more user-state metrics includes a user productivity metric indicating a level of user productivity based at least on computing information generated by one or more productivity application programs.

5. The computing system of claim 2, wherein the one or more user-state metrics includes a camera usage metric indicating a level of camera usage during user interactions facilitated by one or more personal communication application programs.

6. The computing system of claim 2, wherein the one or more user-state metrics includes an empathy metric indicating a level of user empathy based at least on user communication information received from one or more personal communication application programs.

7. The computing system of claim 6, wherein the communication information includes one or more email messages, text messages, audio transcripts, and/or comments posted in communication application programs and/or productivity application programs.

8. The computing system of claim 6, wherein the communication information includes a user audio segment, and wherein the empathy metric is determined based at least on voice patterns detected in the user audio segment.

9. The computing system of claim 2, wherein the one or more user-state metrics includes a facial expression metric indicating a user well being state based at least on images captured, via a camera, during user interactions facilitated by one or more personal communication application programs.

10. The computing system of claim 2, wherein the one or more user-state metrics includes a location metric indicating a level to which a user's location changes on an interaction-to-interaction basis when interacting with one or more of a personal communication application program and a productivity application program.

11. The computing system of claim 1, wherein computing information attributed to the user account is received from one or more personal communication application programs and one or more productivity application programs.

12. The computing system of claim 1, wherein the user account includes a social network graph including a plurality of different users, wherein the mitigation machine is configured to, for each different user in the social network graph, determine an interaction quality indicating a level to which the different user influences the user well being state during user interactions, and wherein the mitigation operation includes sending, to the user computer via the network communication subsystem, an invitation notification recommending user interaction with a different user in the social network graph that has a positive interaction quality.

13. The computing system of claim 12, wherein the mitigation operation includes sending, via the network communication subsystem, to a different user computer corresponding to the different user in the social network graph that has a positive interaction quality, a wellness check notification recommending the different user initiate user interaction.

14. The computing system of claim 12, wherein the mitigation operation includes at least temporarily suppressing notifications from a different user in the social network graph that has a negative interaction quality.

15. The computing system of claim 1, wherein the mitigation operation includes sending, to the user computer via the network communication subsystem, a benefits reminder notification indicating well being state improving benefits that are currently available for the user account.

16. A computer-implemented method, comprising:
receiving, via a network communication subsystem, computing information from a user computer;
attributing, via an attribution machine, the computing information to a user account;
progressively updating, via a well being assessment machine, a well being score over time for the user account based at least on the computing information; and
performing, via a mitigation machine, a mitigation operation associated with the user account based at least on an above threshold rate of decrease of the well being score.

17. The computer-implemented method of claim 16, wherein the user account includes a social network graph including a plurality of different users, wherein the mitigation machine is configured to, for each different user in the social network graph, determine an interaction quality indicating a level to which the different user influences the user well being state during user interactions, and wherein the mitigation operation includes sending, to the user computer via the network communication subsystem, an invitation notification recommending user interaction with a different user in the social network graph that has a positive interaction quality.

18. The computer-implemented method of claim 17, wherein the mitigation operation includes sending, via the network communication subsystem, to a different user computer corresponding to the different user in the social network graph that has a positive interaction quality, a wellness check notification recommending the different user initiate user interaction.

19. The computer-implemented method of claim 17, wherein the mitigation operation includes at least temporarily suppressing notifications from a different user in the social network graph that has a negative interaction quality.

20. A computing system, comprising:
a network communication subsystem configured to communicate with a plurality of user computers;
an attribution machine configured to attribute, to a user account, computing information that the network communication subsystem receives from a user computer of the plurality of user computers, the computing information including one or more user-state metrics output from one or more machine-learning models executing on the user computer from which the computing information is received;
a well being assessment machine configured to progressively update a well being score over time for the user account based at least on the user-state metrics; and
a mitigation machine configured to perform a mitigation operation associated with the user account based at least on an above threshold rate of decrease of the well being score.
